(19) United States
(12) Patent Application Publication
(10) Pub. No.: US 2025/0082287 A1
(43) Pub. Date: Mar. 13, 2025

(54) DENTAL X-RAY DEVICE

(71) Applicant: TAKARA BELMONT CORPORATION, Osaka (JP)

(72) Inventors: Shigeaki YASUI, Osaka (JP); Hitoshi YOSHIDA, Osaka (JP); Munehiro KODAMA, Osaka (JP)

(21) Appl. No.: 18/030,184

(22) PCT Filed: Oct. 7, 2020
(86) PCT No.: PCT/JP2020/038040

§ 371 Date: Apr. 4, 2023

(51) Int. Cl. A61B 6/00; A61B 1/00; A61B 1/06; A61B 1/24; A61B 6/46; A61B 6/51

(52) U.S. Cl. CPC A61B 6/4417; A61B 1/06; A61B 1/24; A61B 6/4458; A61B 6/51; A61B 1/00045; A61B 6/461

(57) ABSTRACT

To provide a dental X-ray device compact enough for use in a treatment room, a dental X-ray device includes: an X-ray generator source; an X-ray emitter section configured to emit X rays that the X-ray generator source has generated; a body having a surface on which the X-ray emitter section is disposed and containing the X-ray generator source; a cone continuous with the X-ray emitter section and defining a field of irradiation with the X ray; and a dental lighting section provided for the body and including a light emitter.

### TECHNICAL FIELD

The present invention relates to dental X-ray devices.

### BACKGROUND ART

Dental practice is performed in a treatment room equipped mainly with a dental chair and various instruments. Dental treatment may involve radiographic image diagnosis with use of a dental X-ray device configured for intraoral radiography (hereinafter referred to simply as “dental X-ray device”). Intraoral radiography involves, for example, the use of a dental X-ray device and an X-ray detector (for example, a sensor or a film) held in the oral cavity of a patient. Patent Literature 1 discloses an example dental X-ray device. As disclosed in Patent Literature 1, a dental X-ray device (“radiography device” in Patent Literature 1) includes an X-ray generator unit (“head”), a cone continuous with the X-ray generator unit, and an arm configured to hold the X-ray generator unit and allowing the X-ray generator unit to be moved freely for a site targeted for radiography. Such a dental X-ray device is typically disposed in an X-ray room separate from a treatment room.

### CITATION LIST

### Patent Literature

### Patent Literature 1

Japanese Unexamined Patent Application Publication, Tokukai, No. 2015-112391

### SUMMARY OF INVENTION

### Technical Problem

Dental practice may involve use of a dental X-ray device in a treatment room under a condition, for instance, if the treatment room satisfies a particular requirement such as the treatment room being equipped with only one dental chair. The use of a dental X-ray device in a treatment room requires equipment for protection against X rays inside the treatment room. Such use, on the other hand, eliminates the need to create a separate X-ray room, thereby allowing efficient use of the treatment space, and also eliminates the need for a patient to move to an X-ray room during the treatment, thereby allowing radiography of the teeth of the patient during a therapy such as root canal therapy while the patient is sitting on a dental chair. However, treatment rooms contain, for example, a tray table, instrument holder, and dental lighting device for a dentist around a dental chair. The dental lighting device includes a lighting section held by an arm. Disposing a dental X-ray device in such a treatment room results in the treatment room additionally containing the X-ray generator unit of the dental X-ray device and an arm for holding the X-ray generator unit. This means that the treatment room ends up containing a plurality of arms and the like for holding different devices. This may prevent the dentist from conveniently operating different devices such as the dental X-ray device. Further, treatment rooms may lack ample space around the dental chair with the dental chair reclined. There is thus room for improvement of a dental X-ray device for use in a treatment room.

The above circumstances have led to a demand for a dental X-ray device compact enough for use in a treatment room.

### Solution to Problem

A dental X-ray device according to the present invention characteristically includes: an X-ray generator source; an X-ray emitter section configured to emit X rays that the X-ray generator source has generated; a body having a surface on which the X-ray emitter section is disposed and containing the X-ray generator source; a cone continuous with the X-ray emitter section and defining a field of irradiation with the X ray; and a dental lighting section provided for the body and including a light emitter.

A typical dental X-ray device includes an X-ray generator unit configured for use while being held by a movable arm. With a dental X-ray device in a treatment room, a person may be obstructed by, for example, a dental lighting section and an arm holding it. In view of that, the dental X-ray device configured as above includes a dental lighting section on its body. The above configuration thus requires only one arm for holing both the body and the dental lighting section, and eliminates the need to include separate holder arms. This saves space in a treatment room, and allows effective use of the space around the dental chair. This in turn allows a person to appropriately perform treatment and operate various devices including the dental X-ray device in the treatment room.

The dental X-ray device is further characteristically configured such that the dental lighting section is oriented such that the dental lighting section emits light with an optical axis parallel to an irradiation direction that forms an angle of more than 90 degrees with an emission direction parallel to a principal ray among the X rays emitted from the X-ray emitter section.

Typical dental treatment does not involve emitting X rays to the teeth of a patient while irradiating the teeth of the patient with light. With the dental X-ray device configured as above, the lighting section is not directed to the teeth of a patient when the X-ray emitter section emits X rays to the teeth of the patient, while the X-ray emitter section is not directed to the teeth of a patient when the dental lighting section irradiates the teeth of the patient with light. The dental X-ray device may, for instance, be configured such that the dental lighting section emits light with an optical axis parallel to an irradiation direction that forms an angle of 180 degrees with an emission direction parallel to the principal ray among X rays emitted from the X-ray emitter section. In this case, the X-ray emitter section is not directed to a patient while the dental lighting section is directed to the patient. The dental X-ray device thus prevents a patient from being exposed to X rays accidentally emitted from the X-ray emitter section while the dental lighting section is irradiating the patient with light, thereby ensuring safety for the patient. Further, the dental X-ray device may be configured such that the X-ray emitter section alone is disposed at one end and that the lighting section alone is disposed at the other end. This reduces the area of the surface that faces the patient during dental treatment, and allows the dental X-ray device to be compact.

The dental X-ray device is further characteristically configured such that the dental lighting section is oriented such that the dental lighting section emits light with an optical axis parallel to an irradiation direction that forms an angle of 90 degrees with an emission direction parallel to a principal ray among the X rays emitted from the X-ray emitter section.

With the above configuration, the dental lighting section does not irradiate a patient with light when the X-ray emitter section emits X rays to the teeth of the patient, while the X-ray emitter section does not emit X rays to the teeth of a patient when the dental lighting section irradiates the patient with light. Further, the dental X-ray device is configured such that simply rotating the body by 90 degrees about an axis perpendicular to the principal ray among X rays and the optical axis of light allows a switch between X-ray emission from the X-ray emitter section and light emission by the dental lighting section. This in turn allows easy use of the dental X-ray device.

The dental X-ray device characteristically further includes: an arm configured to hold the body in such a manner that the body is rotatable, wherein the dental lighting section is rotatable by not less than 90 degrees relative to the arm.

With the above configuration, the body is held by an arm in such a manner that the body is rotatable, and the dental lighting section is rotatable by not less than 90 degrees relative to the arm. This allows the orientation of the body to be changed easily, and thereby allows the body to easily switch between X-ray emission from the X-ray emitter section and light emission by the dental lighting section. This in turn allows easy use of the dental X-ray device.

The dental X-ray device is further characteristically configured such that the dental lighting section is oriented such that the dental lighting section emits light with an optical axis parallel to an irradiation direction that forms an angle of less than 90 degrees with an emission direction parallel to a principal ray among the X rays emitted from the X-ray emitter section.

With the dental X-ray device configured as above, simply rotating the body by less than 90 degrees easily switches between X-ray emission from the X-ray emitter section and light emission by the dental lighting section. This in turn allows easier use of the dental X-ray device.

The dental X-ray device is further characteristically configured such that the light emitter is proximate to the X-ray emitter section.

With the above configuration, the light emitter of the dental lighting section is proximate to the X-ray emitter section. This allows the dental lighting section to emit light with an optical axis parallel to an irradiation direction that forms an angle of close to 0 degrees with an emission direction parallel to a principal ray among the X rays emitted from the X-ray emitter section. The above configuration allows the dental X-ray device to emit either X rays or light through a switch between (i) X-ray emission from the X-ray emitter section and (ii) light emission from the dental lighting section without rotation of the body. This in turn allows easier use of the dental X-ray device.

The dental X-ray device is further characteristically configured such that the X-ray emitter section does not emit the X rays while the dental lighting section is emitting light.

Typical dental treatment does not involve emitting X rays to a patient while irradiating the patient with light. If, however, a dental X-ray device includes a dental lighting section, and an X-ray generator unit (specifically, an X-ray generator source, an X-ray emitter section, or the like) is accidentally operated while the dental lighting section is irradiating a patient with light, the X-ray emitter section may emit X rays. In view of that, the dental X-ray device is configured such that the X-ray emitter section does not emit the X rays while the dental lighting section is emitting light. The dental X-ray device thereby appropriately ensures safety for the patient.

The dental X-ray device is further characteristically configured such that the cone is detachable from the body.

With the above configuration, the cone, which is detachable from the body, may be detached from the body when no X-ray emission is needed. This allows the dental X-ray device to be compact, and in turn allows the body to be manipulated easily for a change of its orientation. In a case where the X-ray emitter section and the dental lighting section are on the same side, and the dental lighting section emits light with the cone continuous with the body, the cone would block the light. Detaching the cone from the body prevents such an inconvenience caused by the cone.

The dental X-ray device is further characteristically configured such that the cone is capable of extension and contraction.

With the above configuration, the cone, which is capable of extension and contraction, may be contracted when no X-ray emission is needed. This allows the body to be manipulated easily for a change of its orientation. In a case where the X-ray emitter section and the dental lighting section are on the same side, and the dental lighting section emits light with the cone continuous with the body, the cone would block the light. Contracting the cone prevents such an inconvenience caused by the cone.

The dental X-ray device is further characteristically configured such that the cone is collapsible.

With the above configuration, the cone, which is collapsible, may be collapsed when no X-ray emission is needed. This allows the body to be manipulated easily for a change of its orientation. In a case where the X-ray emitter section and the dental lighting section are on the same side, and the dental lighting section emits light with the cone continuous with the body, the cone would block the light. Collapsing the cone prevents such an inconvenience caused by the cone.

The dental X-ray device characteristically further includes: a display section provided for the body.

With the above configuration, the body is provided with a display section that allows various pieces of information on a patient to be easily learned about.

The dental X-ray device is further characteristically configured such that the display section has a visual recognition range overlapping with an area with which the dental lighting section irradiates with light.

With the above configuration, the display section has a visual recognition range overlapping with an area with which the dental lighting section irradiates with light. This facilitates seeing, for example, various pieces of information on the patient on the display section while the dental lighting section is in use.

The dental X-ray device characteristically further includes: a visible light image capture section configured to capture an image of a site on which the X rays emitted from the X-ray emitter section are incident.

The above configuration allows the visible light image capture section to capture an image of a site on which the X rays are incident (that is, the oral cavity of a patient including the teeth and its surroundings). Viewing on a monitor or the like an image captured by the visible light image capture section facilitates learning about how the dental X-ray device is positioned relative to the patient. This means that an image captured by the visible light image capture section helps to place the dental X-ray device at an appropriate position proximate to the oral cavity of the patient.

The dental X-ray device characteristically further includes: a visible light image capture section configured to capture an image of a site on which the X rays emitted from the X-ray emitter section are incident, wherein the display section is configured to display the image.

The above configuration allows the visible light image capture section to capture an image of a site on which the X rays are incident (that is, the oral cavity of a patient including the teeth and its surroundings). Such an image captured by the visible light image capture section facilitates learning about how the dental X-ray device is positioned relative to the patient. This means that an image captured by the visible light image capture section helps to place the dental X-ray device at an appropriate position proximate to the oral cavity of the patient. Further, the display section on the body facilitates viewing an image captured by the visible light image capture section.

### BRIEF DESCRIPTION OF DRAWINGS

FIG. 1 is a general perspective diagram illustrating use of an X-ray generator unit of a dental X-ray device.

FIG. 2 is a general perspective diagram illustrating use of a lighting unit of a dental X-ray device.

FIG. 3 is a perspective diagram illustrating a lighting unit of a dental X-ray device as Embodiment 1.

FIG. 4 is a perspective diagram illustrating an X-ray emitter section of a dental X-ray device as Embodiment 1.

FIG. 5 is a perspective diagram illustrating use of an X-ray generator unit of a dental X-ray device as Embodiment 1.

FIG. 6 is a side diagram illustrating how an X-ray emitter section and dental lighting section of a dental X-ray device as Embodiment 1 are positioned relative to each other.

FIG. 7 is a perspective diagram illustrating use of a lighting unit of a dental X-ray device as Variation 1 of Embodiment 1.

FIG. 8 is a perspective diagram illustrating use of an X-ray generator unit of a dental X-ray device as Variation 1 of Embodiment 1.

FIG. 9 is a perspective diagram illustrating use of a lighting unit of a dental X-ray device as Variation 2 of Embodiment 1.

FIG. 10 is a perspective diagram illustrating use of an X-ray generator unit of a dental X-ray device as Variation 2 of Embodiment 1.

FIG. 11 is a perspective diagram illustrating use of a lighting unit of a dental X-ray device as Embodiment 2.

FIG. 12 is an exploded perspective diagram illustrating a dental X-ray device as Embodiment 2.

FIG. 13 is a perspective diagram illustrating use of an X-ray generator unit of a dental X-ray device as Embodiment 2.

FIG. 14 is a perspective diagram illustrating use of a lighting unit of a dental X-ray device as Embodiment 3.

FIG. 15 is a perspective diagram illustrating use of an X-ray generator unit of a dental X-ray device as Embodiment 3.

FIG. 16 is a side diagram illustrating how an X-ray emitter section and dental lighting section of a dental X-ray device as Embodiment 3 are positioned relative to each other.

FIG. 17 is a perspective diagram illustrating use of a lighting unit of a dental X-ray device as Embodiment 4.

FIG. 18 is an exploded perspective diagram illustrating a dental X-ray device as Embodiment 4.

FIG. 19 is a perspective diagram illustrating use of an X-ray generator unit of a dental X-ray device as Embodiment 4.

FIG. 20 is a perspective diagram illustrating use of a lighting unit of a dental X-ray device as Embodiment 5.

FIG. 21 is a rear perspective diagram illustrating a dental X-ray device as Embodiment 5.

FIG. 22 is a perspective diagram illustrating use of an X-ray generator unit of a dental X-ray device as Embodiment 5.

FIG. 23 is a side diagram illustrating how an X-ray emitter section and dental lighting section of a dental X-ray device as Embodiment 5 are positioned relative to each other.

FIG. 24 is a perspective diagram illustrating a dental X-ray device as a variation of Embodiment 5.

FIG. 25 is a diagram illustrating a cone of a dental X-ray device as an alternative embodiment.

FIG. 26 is a diagram illustrating a cone of a dental X-ray device as an alternative embodiment.

### DESCRIPTION OF EMBODIMENTS

The description below deals with dental X-ray devices as embodiments of the present invention with reference to drawings. The description below uses the same reference sign for the same element, and avoids dealing with elements with the same reference sign more than once.

### Embodiment 1

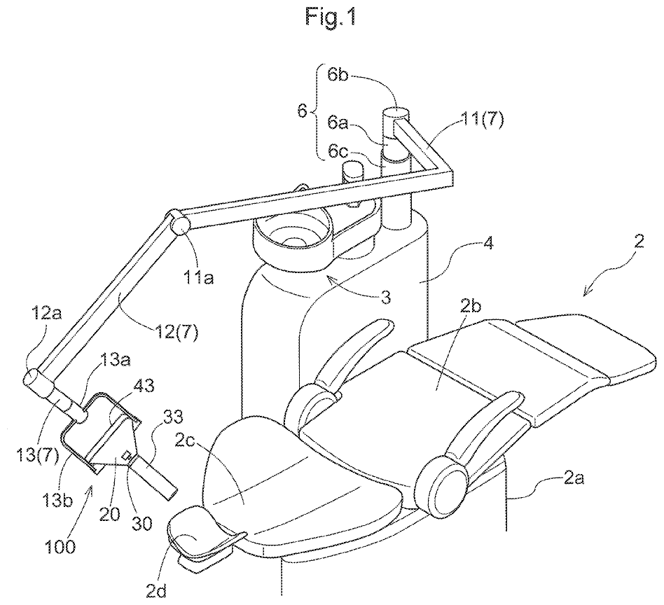

FIGS. 1 to 6 each illustrate a dental X-ray device 100 for intraoral radiography. The dental X-ray device 100 is configured to emit X rays to an X-ray detector such as an X-ray film or an imaging plate held in the oral cavity of a patient to radiograph the teeth of the patient.

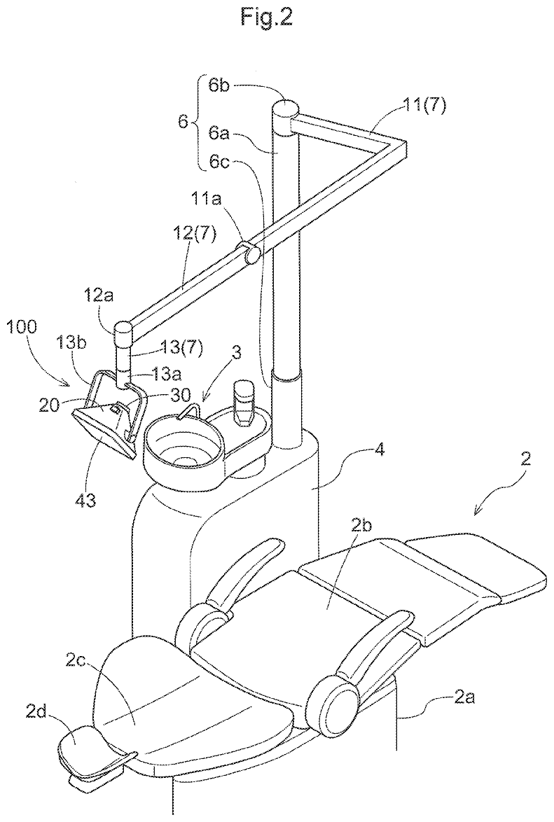

As illustrated in FIGS. 1 and 2, the dental X-ray device 100 is disposed in a treatment room. The treatment room contains, for example, a dental chair 2, a water supplier device (spittoon device) 3, and an instrument holder (not shown). The dental chair 2 includes a base 2a, a seating plate 2b supported by the base 2a, a back plate 2c rotatable about an end of the seating plate 2b, and a headrest 2d attached to the back plate 2c. The water supplier device 3 is on a base unit 4 provided with a supportive column 6 having an upper end portion 6b with which an arm 7 is connected at an end thereof. The dental X-ray device 100 as the present embodiment is held by the arm 7.

FIG. 1 illustrates how an X-ray generator unit 30 (described later) is used as provided for the dental X-ray device 100. FIG. 2 illustrates how a lighting unit 43 (described later) is used as provided for the dental X-ray device 100. The dental X-ray device 100 may be oriented for X-ray emission by the X-ray generator unit 30, in which case the dental X-ray device 100 is proximate to the dental chair 2 by means of arm sections 11 to 13. The dental X-ray device 100 emits X rays to a patient sitting on the dental chair 2 or lying on their back on the dental chair 2. The dental X-ray device 100 may be oriented for light emission by the lighting unit 43, in which case the dental X-ray device 100 is apart from the dental chair 2 upward by a distance by means of the arm sections 11 to 13.

The dental X-ray device 100 is held by the supportive column 6 and the arm 7. The supportive column 6 has a circular cross section perpendicular to its longitudinal direction. The supportive column 6 includes an extendable portion 6a, an upper end portion 6b at a leading end of the extendable portion 6a, and a support portion 6c supporting the extendable portion 6a. The extendable portion 6a is capable of vertical extension and contraction relative to the support portion 6c. The upper end portion 6b is rotatable relative to the extendable portion 6a. The arm 7 extends from the upper end portion 6b of the supportive column 6 in a direction perpendicular to the supportive column 6. The arm 7 includes a first arm section 11, a second arm section 12, and a third arm section 13. The first arm section 11 includes (i) a base end portion attached to the upper end portion 6b of the supportive column 6 and (ii) a leading end portion 11a opposite to the base end portion. The first arm section 11 extends horizontally, and is rotatable about the axis of the supportive column 6 integrally with the upper end portion 6b. The second arm section 12 includes a base end portion attached to the leading end portion 11a of the first arm section 11. The second arm section 12 is rotatable about an axis perpendicular to the supportive column 6. The third arm section 13 is attached to the leading end portion 12a of the second arm section 12. The third arm section 13 includes a bar 13a extending from the second arm section 12 perpendicularly thereto and a pair of holder arms 13b attached to the leading end portion of the bar 13a. The dental X-ray device 100 is rotatably attached to the respective leading ends of the holder arms 13b. The arm sections 11 to 13 are an example means for supporting the dental X-ray device 100.

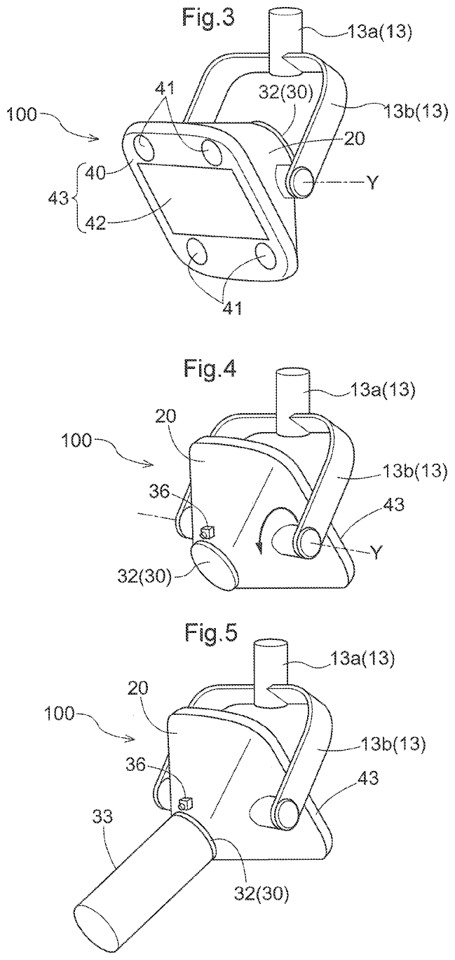

As illustrated in FIGS. 3 to 6, the dental X-ray device 100 includes a body 20, an X-ray generator unit 30, and a dental lighting section 40 (hereinafter referred to also as “lighting section 40”). The body 20 is a casing entirely with the shape of a truncated square pyramid. The body 20 has an opening 21 at the top surface with a smaller area, and has a bottom surface with a larger area to serve as an attachment surface 22. The X-ray generator unit 30 includes an X-ray emitter section 32 at the opening 21. The dental X-ray device 100 includes on the attachment surface 22 a lighting unit 43 including the lighting section 40 and a display section 42 integrally with each other.

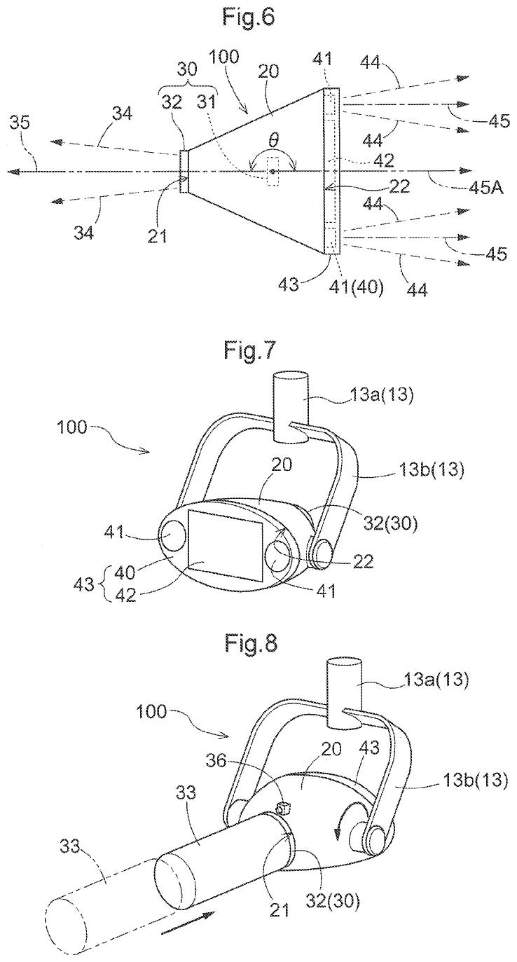

The X-ray generator unit 30 is disposed on the body 20, and includes an X-ray generator source 31 (for example, an X-ray tube) as well as the X-ray emitter section 32. As illustrated in FIG. 6, the X-ray generator source 31 is disposed in the body 20 and configured to generate X rays 34 for emission through the opening 21 from the X-ray emitter section 32. The X-ray emitter section 32 is disposed on a surface of the body 20. As illustrated in FIG. 4, the dental X-ray device 100 includes an X-ray emitter section 32 in the shape of a flat surface. The X-ray emitter section 32 may alternatively be in the shape of an outwardly curved surface. As illustrated in FIG. 5, the dental X-ray device 100 includes a cone (X-ray emission cylinder) 33 configured to be continuous with the X-ray emitter section 32 and define the field of irradiation with the X rays 34. The cone 33 allows X rays 34 from the X-ray emitter section 32 to be directed to a desired position in the oral cavity of the patient. As illustrated in FIGS. 4 and 5, the cone 33 for the present embodiment is detachably attachable to the body 20 (specifically, to the X-ray emitter section 32). The cone 33, which is detachably attachable to the body 20, may be detached from the body 20 when no X-ray emission is needed. This allows the dental X-ray device 100 to be compact, and in turn allows the body 20 to be manipulated easily for a change of its orientation. The cone 33 is attachable to and detachable from the body 20 with, for instance, the X-ray emitter section 32 having an engagement groove (not shown) along its outer circumferential surface and the cone 33 having on its inner circumferential surface an engagement nail (not shown) capable of engaging with and disengaging from the engagement groove.

The lighting unit 43 is attached to the attachment surface 22 of the body 20 in such a manner as to be in contact with the entire attachment surface 22. As illustrated in FIG. 3, the display section 42 is rectangular, and is disposed at a central portion of the lighting unit 43. The display section 42 is in the form of, for example, a liquid crystal monitor, and serves to display, for example, (i) images captured by a camera 36 (described later) of the oral cavity of the patient and its surroundings and (ii) various pieces of information on the patient. The lighting section 40 includes a plurality of (four for the present embodiment) light emitters 41 proximate respectively to the four corners of the display section 42. The light emitters 41 are each in the form of, for example, a halogen light or an LED light. The lighting section 40 may further include a reflector plate (not shown) around the light emitters 41 and/or a transparent cover (not shown) on the front side of the light emitters 41 and the display section 42. The light emitters 41 emit light 44 (see FIG. 6) to the face of the patient for appropriate dental treatment.

The dental X-ray device 100 includes a body 20 provided with an X-ray generator unit 30 and a lighting section 40 of a lighting unit 43 as described above. The arm sections 11 to 13 serve as a holder arm for both the X-ray generator unit 30 and the lighting section 40, and eliminates the need to include separate holder arms. This saves space in a treatment room, and allows effective use of the space around the dental chair 2. This in turn allows a person to appropriately perform treatment and operate various devices including the dental X-ray device 100 in the treatment room.

As illustrated in FIG. 6, the lighting section 40 is oriented such that the light emitters 41 each emit light 44 with an optical axis 45 parallel to an irradiation direction (imaginary line 45A) that forms an angle θ of more than 90 degrees and not more than 180 degrees with an emission direction parallel to the principal ray 35 among X rays 34 emitted from the X-ray emitter section 32. The light emitters 41 for the present embodiment are oriented such that the angle θ is 180 degrees.

Typical dental treatment does not involve emitting X rays 34 to a patient while irradiating the patient with light 44. With the dental X-ray device 100 configured as above, the lighting section 40 is not directed to the teeth of a patient when the X-ray emitter section 32 emits X rays 34 to the teeth of the patient, while the X-ray emitter section 32 is not directed to the teeth of a patient when the lighting section 40 irradiates the teeth of the patient with light 44. The present embodiment is configured such that the lighting section 40 emits light 44 with an optical axis 45 parallel to an irradiation direction that forms an angle θ of 180 degrees with an emission direction parallel to the principal ray 35 among X rays 34 emitted from the X-ray emitter section 32. In this case, the X-ray emitter section 32 is not directed to a patient while the lighting section 40 is directed to the patient. The dental X-ray device 100 thus prevents a patient from being exposed to X rays 34 accidentally emitted from the X-ray emitter section 32 while the lighting section 40 is irradiating the patient with light 44, thereby ensuring safety for the patient. The dental X-ray device 100 as the present embodiment is configured such that the X-ray emitter section 32 alone is disposed at the opening 21 in the body 20 and that the lighting unit 43 (lighting section 40) alone is disposed on the attachment surface 22. This reduces the area of the surface that faces the patient during dental treatment, and allows the dental X-ray device 100 to be compact.

FIG. 3 illustrates the dental X-ray device 100 with the lighting section 40 in use. The third arm section 13 includes a pair of holder arms 13b configured to hold the body 20 in such a manner that the body 20 is rotatable about a rotation axis Y. The holder arms 13b are example arms for holding the body 20 in such a manner that the body 20 is rotatable. The body 20 is rotatable about the rotation axis Y while being held by the holder arms 13b. The body 20 as illustrated in FIG. 3 is rotated about the rotation axis Y by 180 degrees for use of the X-ray generator unit 30 of the dental X-ray device 100 for radiography. This causes the X-ray emitter section 32 on the body 20 to face the dental chair 2 on which a patient is sitting, as illustrated in FIG. 4. Then, as illustrated in FIG. 5, the cone 33 is attached to the X-ray emitter section 32, and the body 20 is moved by means of, for example, the arm sections 11 to 13 so that the leading end of the cone 33 is proximate to the oral cavity of the patient. This renders the dental X-ray device 100 ready for radiography.

As illustrated in FIGS. 4 and 5, the body 20 is provided with a camera 36 (which is an example of the “visible light image capture section”) at a predetermined position proximate to the X-ray emitter section 32. The camera 36 is configured to capture an image of a site on which X rays 34 from the X-ray emitter section 32 are incident (that is, the oral cavity of a patient including the teeth and its surroundings). The display section 42 is configured to display the image captured by the camera 36. Viewing on a monitor or the like an image captured by the camera 36 facilitates learning about how the dental X-ray device 100 is positioned relative to the patient. This means that an image captured by the camera 36 helps to place the dental X-ray device 100 at an appropriate position proximate to the oral cavity of the patient. Further, the display section 42 on the body 20 facilitates viewing an image captured by the camera 36.

As illustrated in FIGS. 3 and 6, the display section 42 has a visual recognition range that overlaps with the area with which the lighting section 40 irradiates with light 44. This facilitates seeing, for example, various pieces of information on the patient on the display section 42 while the lighting section 40 is in use.

### Variation 1 of Embodiment 1

Embodiment 1 has Variation 1 in which as illustrated in FIGS. 7 and 8, the dental X-ray device 100 includes a body 20 in the shape of a circular truncated cone having an oval bottom surface and facing laterally. The body 20 has a bottom surface with a larger area to serve as an attachment surface 22 on which a lighting unit 43 is disposed. The body 20 also has at the top surface with a smaller area an opening 21 at which an X-ray emitter section 32 is disposed. FIG. 7 illustrates a lighting section 40 in use. The lighting unit 43 includes a rectangular display section 42 at a central portion. The lighting unit 43 also includes a lighting section 40 including a plurality of (two for the present embodiment) light emitters 41 on the opposite sides of the display section 42. For radiography with use of an X-ray generator unit 30, the body 20 as illustrated in FIG. 7 is rotated by 180 degrees and provided with a cone 33 on the X-ray emitter section 32, as illustrated in FIG. 8. The body 20 is provided with a camera 36 at a predetermined position proximate to the X-ray emitter section 32 as illustrated in FIG. 8.

### Variation 2 of Embodiment 1

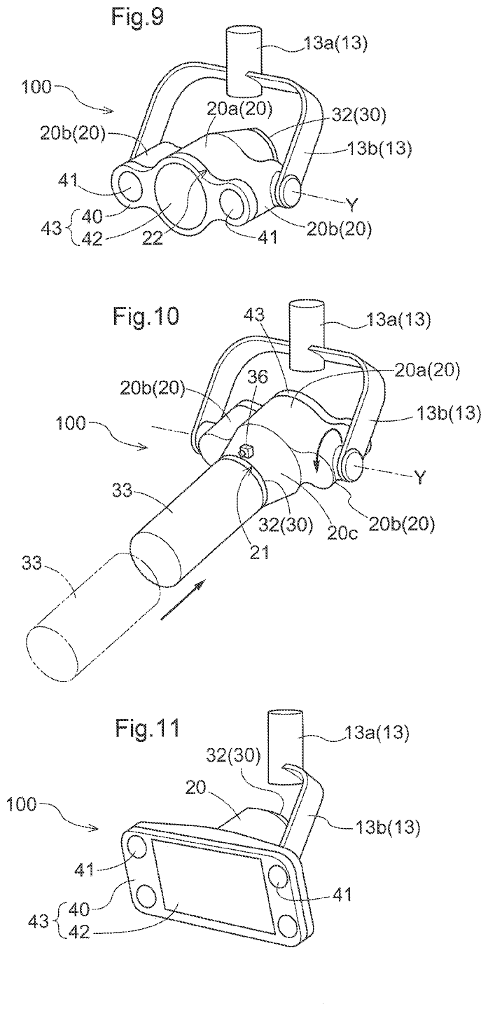

Embodiment 1 has Variation 2 in which as illustrated in FIGS. 9 and 10, the dental X-ray device 100 includes a body 20 including (i) a large cylindrical column portion 20a at a lateral center and (ii) a pair of small cylindrical column portions 20b and 20b smoothly contiguous with the respective opposite sides of the large cylindrical column portion 20a. The large cylindrical column portion 20a and the small cylindrical column portions 20b and 20b have respective central axes on a single plane. The body 20 has an attachment surface 22 made up of a flush arrangement of a surface of the large cylindrical column portion 20a and a surface of each of the small cylindrical column portions 20b and 20b. The large cylindrical column portion 20a protrudes further than the small cylindrical column portions 20b and 20b on the side opposite to the attachment surface 22. The protrusion of the large cylindrical column portion 20a is a tapered protrusion 20c, which is tapered, that is, which becomes gradually narrower toward a leading end. The tapered protrusion 20c has an opening 21 at its leading end. FIG. 9 illustrates the lighting section 40 in use. The lighting unit 43 includes a circular display section 42 at the large cylindrical column portion 20a. The lighting unit 43 also includes a lighting section 40 including a plurality of (two for the present embodiment) light emitters 41 at the small cylindrical column portions 20b and 20b, which are on the opposite sides of the display section 42. FIG. 10 illustrates an X-ray generator unit 30 in use, with an X-ray emitter section 32 at the opening 21. For radiography with use of an X-ray generator unit 30, the body 20 as illustrated in FIG. 9 is rotated by 180 degrees and provided with a cone 33 on the X-ray emitter section 32, as illustrated in FIG. 10. The body 20 is provided with a camera 36 at a predetermined position proximate to the X-ray emitter section 32 as illustrated in FIG. 10.

### Embodiment 2

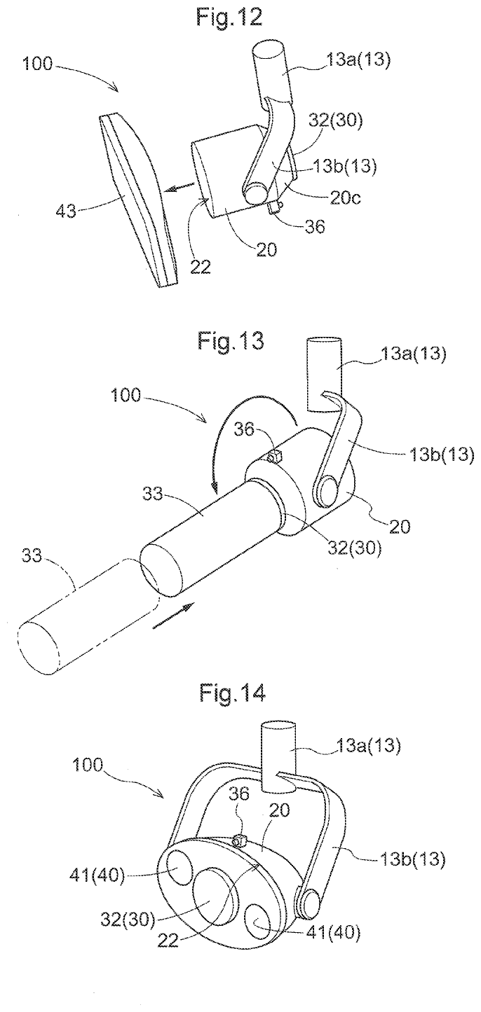

Embodiment 2 is, as illustrated in FIGS. 11 and 12, a dental X-ray device 100 including a body 20, an X-ray generator unit 30, and a rectangular lighting unit 43. The present embodiment includes a single holder arm 13b. The body 20 includes at a leading end thereof a tapered protrusion 20c, which is tapered, that is, which becomes gradually narrower toward the leading end. The body 20 is provided with an X-ray emitter section 32 at the leading end of the tapered protrusion 20c. The lighting unit 43 includes a rectangular display section 42 at a central portion. The lighting section 40 includes a plurality of (four for the present embodiment) light emitters 41 proximate respectively to the four corners of the display section 42. FIG. 11 illustrates the lighting unit 43 in use. FIG. 12 illustrates the dental X-ray device 100 as the present embodiment as being configured such that the lighting unit 43 is detachable from the body 20. For radiography with use of the X-ray generator unit 30, the lighting unit 43 is first detached from the body 20 as illustrated in FIG. 12. Then, the body 20 is rotated by 180 degrees and provided with a cone 33 on the X-ray emitter section 32, as illustrated in FIG. 13. The body 20 is provided with a camera 36 at a predetermined position proximate to the X-ray emitter section 32 as illustrated in FIG. 13. The dental X-ray device 100 as the present embodiment is, as described above, configured such that the lighting unit 43 may be detached before radiography with use of the X-ray generator unit 30. This allows the dental X-ray device 100 to be compact and lightweight, and thereby allows the dental X-ray device 100 to be easily moved to a desired position. The dental X-ray device 100 is also capable of radiography with the lighting unit 43 attached to the body 20.

### Embodiment 3

Embodiment 3 is, as illustrated in FIGS. 14 to 16, a dental X-ray device 100 including a body 20 in the shape of a bullet, that is, with a laterally elongated oval bottom surface at one end and a curved surface at the other end. The present embodiment includes an X-ray emitter section 32 and a lighting section 40 on the same side of the body 20. As illustrated in FIG. 14, the body 20 has at one end an attachment surface 22 with an opening 21 at the center. The body 20 is provided with (i) a circular X-ray emitter section 32 at the opening 21 and (ii) two light emitters 41 as a lighting section 40 on the respective opposite sides of the X-ray emitter section 32. The present embodiment is configured such that as illustrated in FIG. 16, the lighting section 40 is oriented such that the light emitters 41 each emit light 44 with an optical axis 45 parallel to an irradiation direction (imaginary line 45A) that forms an angle θ of less than 90 degrees with an emission direction parallel to the principal ray 35 among X rays 34 emitted from the X-ray emitter section 32. The body 20 is provided with a camera 36 at a predetermined position proximate to the X-ray emitter section 32 as illustrated in FIG. 14.

The present embodiment illustrated in FIGS. 14 to 16 is configured such that the light emitters 41 are proximate to the X-ray emitter section 32 and that the angle θ is 0 degrees. The above configuration allows the dental X-ray device 100 to emit either X rays or light through a switch between (i) X-ray emission from the X-ray emitter section 32 and (ii) light emission by the light emitters 41 of the lighting section 40 without rotation of the body 20. This in turn allows easier use of the dental X-ray device 100.

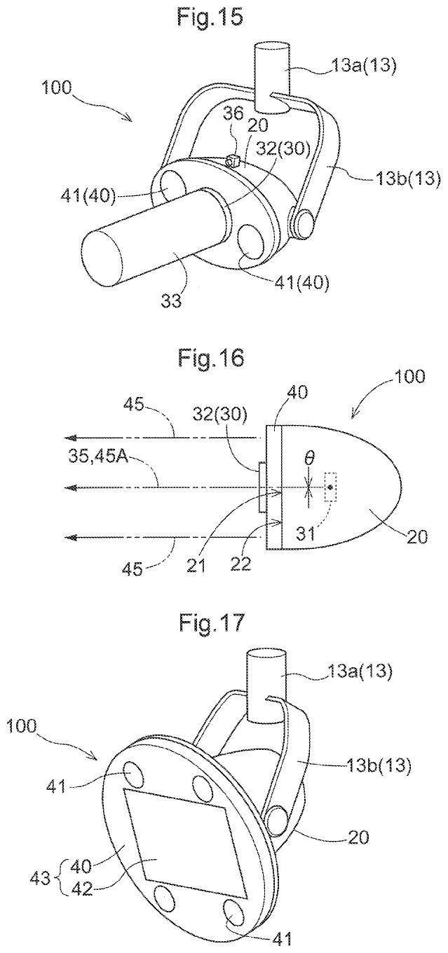

The dental X-ray device 100 as the present embodiment may be provided with a cover or the like (not shown) attachable to the X-ray emitter section 32 to prevent use of the X-ray generator unit 30 when the lighting section 40 is in use. The dental X-ray device 100 may also be configured to, for example, turn off the light emitters 41 to prevent use of the lighting section 40 and be provided with a cone 33 on the X-ray emitter section 32 for radiography with use of the X-ray generator unit 30. This means that the dental X-ray device 100 as illustrated in FIG. 15 does not require the body 20 to be rotated for the X-ray emitter section 32 to emit X rays 34 to a patient. The dental X-ray device 100 is also capable of X-ray emission from the X-ray emitter section 32 and light emission by the lighting section 40 in a simultaneous manner.

### Embodiment 4

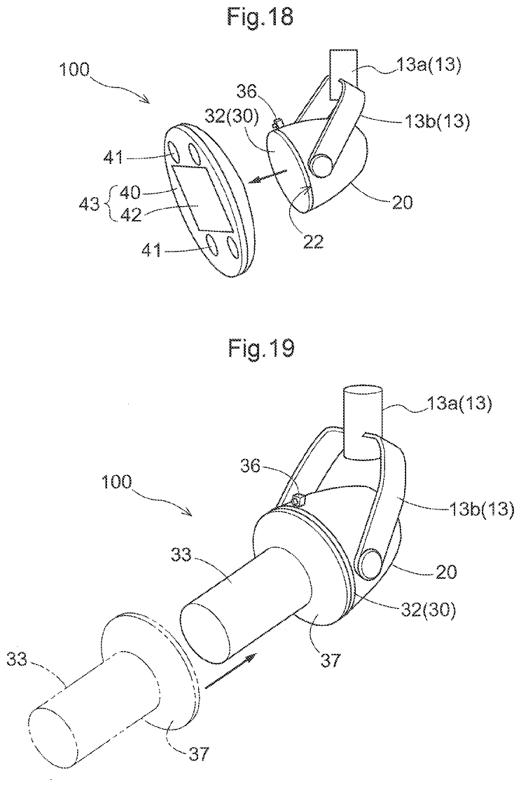

Embodiment 4 is, as illustrated in FIGS. 17 and 18, a dental X-ray device 100 including a body 20, an X-ray generator unit 30, and a disk-shaped lighting unit 43. The dental X-ray device 100 as the present embodiment includes a body 20 in the shape of a bullet with a circular bottom surface. The lighting unit 43 includes a rectangular display section 42 at a central portion. The lighting section 40 includes a plurality of (four for the present embodiment) light emitters 41 proximate respectively to the four corners of the display section 42. As illustrated in FIG. 18, the dental X-ray device 100 as the present embodiment includes an X-ray emitter section 32 at the entire bottom surface. The lighting unit 43 includes a display section 42 and a lighting section 40 on a surface larger than the area of the X-ray emitter section 32. The body 20 is provided with a camera 36 at a predetermined position proximate to the X-ray emitter section 32 as illustrated in FIG. 18. FIG. 17 illustrates the lighting section 40 in use. FIG. 18 illustrates the dental X-ray device 100 as being configured such that the lighting unit 43 is attachable to and detachable from the body 20. For radiography with use of the X-ray generator unit 30, the lighting unit 43 is first detached from the body 20 to expose the X-ray emitter section 32 as illustrated in FIG. 18. Then, the body 20 is provided with a cone 33 on the X-ray emitter section 32 as illustrated in FIG. 19. As described above, the dental X-ray device 100 as the present embodiment requires the lighting unit 43 to be detached for use of the X-ray generator unit 30. This, however, allows the dental X-ray device 100 to be compact and lightweight, and thereby allows the dental X-ray device 100 to be easily moved to a desired position. The cone 33 for the present embodiment includes at an end thereof a collar 37 configured to cover the X-ray emitter section 32 with the cone 33 attached thereto.

### Embodiment 5

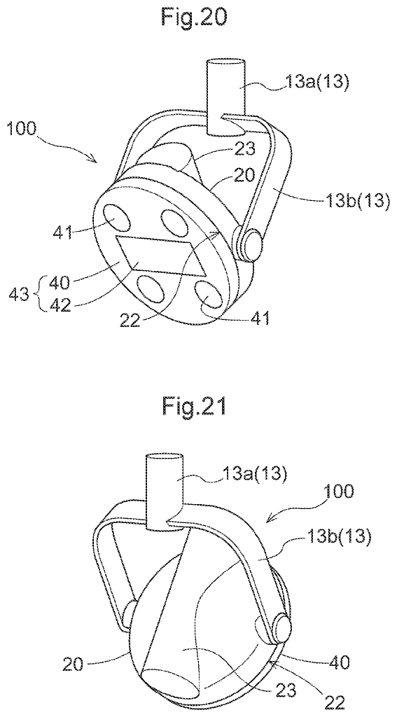

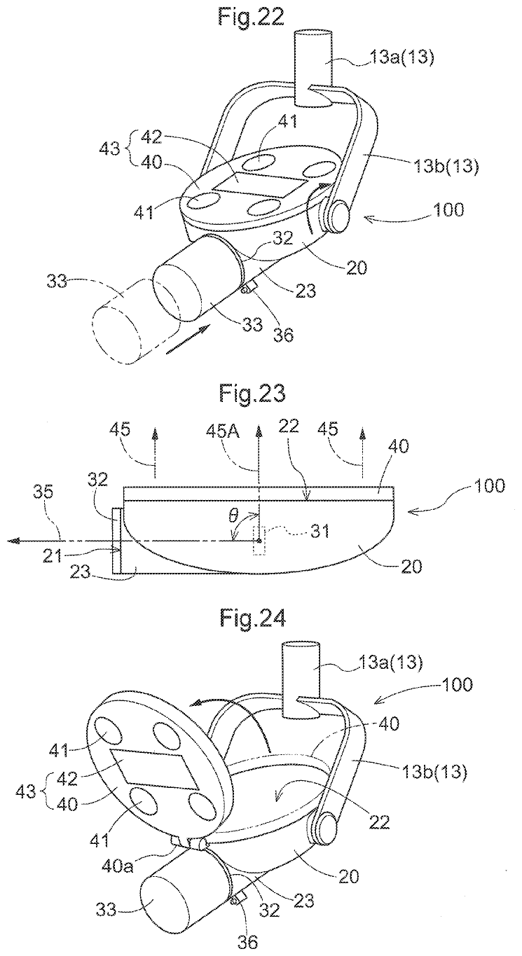

Embodiment 5 is, as illustrated in FIGS. 20 and 21, a dental X-ray device 100 including a body 20, an X-ray generator unit 30, and a disk-shaped lighting unit 43. The body 20 is in the shape of a disk with a flat surface as an attachment surface 22 on one side and a curved surface on the other side. The body 20 is provided with a bottomed cylinder 23 partially embedded in the curved surface and having a central axis parallel to the attachment surface 22. The cylinder 23 has an opening 21, at which the X-ray emitter section 32 is disposed as illustrated in FIG. 23. The lighting unit 43 includes a rectangular display section 42 at a central portion. The lighting section 40 includes a plurality of (four for the present embodiment) light emitters 41 proximate respectively to the four corners of the display section 42. The cylinder 23 is behind the lighting section 40. The present embodiment is configured such that as illustrated in FIG. 23, the lighting section 40 is oriented such that the light emitters 41 each emit light 44 with an optical axis 45 parallel to an irradiation direction (imaginary line 45A) that forms an angle θ of 90 degrees with an emission direction parallel to the principal ray 35 among X rays 34 emitted from the X-ray emitter section 32. The body 20 is provided with a camera 36 at a predetermined position proximate to the X-ray emitter section 32 as illustrated in FIG. 22. FIG. 20 illustrates the lighting section 40 in use. For radiography with use of the X-ray generator unit 30, the body 20 as illustrated in FIG. 20 is rotated by 90 degrees and provided with a cone 33 on the X-ray emitter section 32, as illustrated in FIG. 22. With this configuration, the lighting section 40 does not irradiate a patient with light 44 when the X-ray emitter section 32 emits X rays 34 to the teeth of the patient, while the X-ray emitter section 32 does not emit X rays 34 to the teeth of a patient when the lighting section 40 irradiates the patient with light 44. Further, the dental X-ray device 100 is configured such that simply rotating the body 20 by 90 degrees about an axis perpendicular to the principal ray 35 among X rays 34 and the optical axis 45 of light 44 allows a switch between X-ray emission from the X-ray emitter section 32 and light emission by the lighting section 40. This in turn allows easy use of the dental X-ray device 100.

### Variation of Embodiment 5

Embodiment 5 has a variation in which as illustrated in FIG. 24, the disk-shaped lighting unit 43 (lighting section 40) alone is capable of being oriented differently relative to the body 20. The lighting section 40 includes a hinge 40a proximate to the X-ray emitter section 32 and allowing the lighting section 40 to rotate thereabout. The lighting section 40 is rotatable about the hinge 40a for a switch between a use orientation and a non-use orientation. The dental X-ray device 100 is thereby configured such that changing the orientation of the lighting section 40 alone switches between (i) the use of only either the X-ray generator unit 30 or the lighting section 40 and (ii) the use of both the X-ray generator unit 30 and the lighting section 40. The body 20 is provided with a camera 36 at a predetermined position proximate to the X-ray emitter section 32 as illustrated in FIG. 24.

### Alternative Embodiments

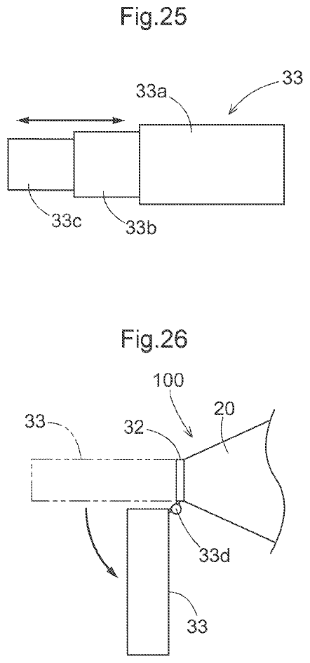

(1) The embodiments described above are each an example including a cone 33 attachable to and detachable from the X-ray emitter section 32 on the body 20. The cone 33 may alternatively be permanently fixed to the X-ray emitter section 32. Further, the cone 33 may be telescopic and capable of extension and contraction in its axial direction, as illustrated in FIG. 25. The example cone 33 illustrated in FIG. 25 includes a first cylinder 33a, a second cylinder 33b, and a third cylinder 33c. The second cylinder 33b has a diameter smaller than the diameter of the first cylinder 33a. The third cylinder 33c has a diameter smaller than the diameter of the second cylinder 33b. For radiography with use of the X-ray generator unit 30, the cone 33 is extended with the second cylinder 33b and the third cylinder 33c taken out of the first cylinder 33a. While the X-ray generator unit 30 is not in use, the first cylinder 33a contains the second cylinder 33b and the third cylinder 33c. The cone 33 illustrated in FIG. 25 may be altered such that the first cylinder 33a has a diameter smaller than the diameter of the second cylinder 33b and that the second cylinder 33b has a diameter smaller than the diameter of the third cylinder 33c. Further, the cone 33 may be collapsible, as illustrated in FIG. 26. The example cone 33 illustrated in FIG. 26 includes a hinge 33d proximate to the X-ray emitter section 32. The cone 33 is rotatable about the hinge 33d to be collapsed into an orientation in which the cone 33 extends along the surface of the X-ray emitter section 32. The cone 33 being attachable to and detachable from the body 20 allows the dental X-ray device 100 to be compact. The telescopic or collapsible cone 33 protrudes from the body 20 in a smaller amount as attached to the X-ray emitter section 32. This allows the orientation of the body 20 to be changed easily, and prevents the cone 33 from accidentally blocking light 44 from the lighting section 40.

(2) Typical dental treatment does not involve emitting X rays 34 to a patient while irradiating the patient with light 44. If, however, the X-ray generator unit 30 (X-ray generator source 31) is accidentally operated while the lighting section 40 is irradiating a patient with light 44, the X-ray emitter section 32 may emit X rays 34. Further, if the dental X-ray device 100 includes an X-ray emitter section 32 and a lighting section 40 on the same side as with Embodiment 3 (see FIGS. 14 to 16), and the X-ray generator unit 30 is accidentally operated while the lighting section 40 is irradiating a patient with light 44, the X-ray emitter section 32 may emit X rays 34 to the patient, leading to an unexpected situation.

The dental X-ray device 100 may, in view of the above, be configured such that the X-ray emitter section 32 will not emit X rays 34 while the lighting section 40 is emitting light 44. To achieve such a configuration, the dental X-ray device 100 may, for instance, include a control section or the like (not illustrated in the drawings) configured to disable the X-ray generator unit 30 from its operation (actuation) while the lighting section 40 is in use. More specifically, the dental X-ray device 100 may, for instance, include a normally open relay circuit configured to be energized upon energization of the lighting section 40 and a control section or the like configured to prevent the X-ray generator unit 30 from being operated (that is, the X-ray generator source 31 from emitting X rays 34) while the relay circuit has a closed contact (that is, while the lighting section 40 is in use). The dental X-ray device 100 may alternatively (i) include a normally closed relay circuit having a contact and configured to be energized upon energization of the lighting section 40 and (ii) be configured such that the X-ray generator unit 30 becomes energized in response to the contact being closed. With this configuration, starting to use the lighting section 40 energizes the relay circuit and opens its contact, which in turn prevents the X-ray generator unit 30 from becoming energized (that is, the X-ray generator source 31 from emitting X rays 34). Further alternatively, the lighting section 40 and the X-ray generator unit 30 (X-ray generator source 31) may be operated with use of respective switches based on a mechanical mechanism that only allows (i) both of the switches to be off or (ii) either of the switches to be on. The dental X-ray device 100 configured as such reliably prevents the X-ray emitter section 32 from emitting X rays 34 if the X-ray generator unit 30 is accidentally operated while the lighting section 40 is emitting light 44. The dental X-ray device 100 thereby appropriately ensures safety for the patient. The dental X-ray device 100 may be configured to selectively allow the X-ray generator unit 30 to emit X rays 34 from the X-ray emitter section 32 or prevent the X-ray generator unit 30 from emitting X rays 34 from the X-ray emitter section 32 while the lighting section 40 is not emitting light 44.

(3) The embodiments described above are each an example including a lighting unit 43 with a display section 42. The dental X-ray device 100 may alternatively not include a display section 42. The embodiments described above are each an example including a lighting section 40 with a plurality of light emitters 41. The lighting section 40 may alternatively include only one light emitter 41. The lighting unit 43 may include a mirror instead of a display section 42.

(4) The embodiments described above are each an example including a body 20 provided with a camera 36 as the visible light image capture section. The body 20 may alternatively be not provided with a camera 36.

### INDUSTRIAL APPLICABILITY

The present invention is widely applicable to dental X-ray devices.

### REFERENCE SIGNS LIST

6 Supportive column
6a Extendable portion
6b Upper end portion
6c Support portion
7 Arm
13b Holder arm
20 Body
20a Large cylindrical column portion
20b Small cylindrical column portion
30 X-ray generator unit
31 X-ray generator source
32 X-ray emitter section
33 Cone
34 X ray
35 Principal ray
36 Camera (visible light image capture section)
40 Dental lighting section
41 Light emitter
42 Display section
43 Lighting unit
44 Light
45 Optical axis
45A Imaginary line
100 Dental X-ray device
Y Rotation axis
θ Angle

1. A dental X-ray device, comprising:
an X-ray generator source;
an X-ray emitter section configured to emit X rays that the X-ray generator source has generated;
a body having a surface on which the X-ray emitter section is disposed and containing the X-ray generator source;
a cone continuous with the X-ray emitter section and defining a field of irradiation with the X ray; and
a dental lighting section provided for the body and including a light emitter, wherein
the dental lighting section is oriented such that the dental lighting section emits light with an optical axis parallel to an irradiation direction that forms an angle of more than 90 degrees with an emission direction parallel to a principal ray among the X rays emitted from the X-ray emitter section.

2. (canceled)

3. A dental X-ray device, comprising:
an X-ray generator source;
an X-ray emitter section configured to emit X rays that the X-ray generator source has generated;
a body having a surface on which the X-ray emitter section is disposed and containing the X-ray generator source;
a cone continuous with the X-ray emitter section and defining a field of irradiation with the X ray; and
a dental lighting section provided for the body and including a light emitter, wherein
the dental lighting section is oriented such that the dental lighting section emits light with an optical axis parallel to an irradiation direction that forms an angle of 90 degrees with an emission direction parallel to a principal ray among the X rays emitted from the X-ray emitter section.

4. The dental X-ray device according to claim 1, further comprising:
an arm configured to hold the body in such a manner that the body is rotatable, wherein
the dental lighting section is rotatable by not less than 90 degrees relative to the arm.

5-6. (canceled)

7. The dental X-ray device according to claim 1, wherein
the X-ray emitter section does not emit the X rays while the dental lighting section is emitting light.

8. The dental X-ray device according to claim 1, wherein
the cone is detachable from the body.

9. The dental X-ray device according to claim 1, wherein
the cone is capable of extension and contraction.

10. The dental X-ray device according to claim 1, wherein
the cone is collapsible.

11. The dental X-ray device according to claim 1, further comprising:
a display section provided for the body.

12. The dental X-ray device according to claim 11, wherein
the display section has a visual recognition range overlapping with an area with which the dental lighting section irradiates with light.

13. The dental X-ray device according to claim 1, further comprising:
a visible light image capture section configured to capture an image of a site on which the X rays emitted from the X-ray emitter section are incident.

14. The dental X-ray device according to claim 11, further comprising:
a visible light image capture section configured to capture an image of a site on which the X rays emitted from the X-ray emitter section are incident, wherein
the display section is configured to display the image.

15. The dental X-ray device according to claim 2, further comprising:
an arm configured to hold the body in such a manner that the body is rotatable, wherein
the dental lighting section is rotatable by not less than 90 degrees relative to the arm.

16. The dental X-ray device according to claim 2, wherein
the X-ray emitter section does not emit the X rays while the dental lighting section is emitting light.

17. The dental X-ray device according to claim 2, wherein
the cone is detachable from the body.

18. The dental X-ray device according to claim 2, wherein
the cone is capable of extension and contraction.

19. The dental X-ray device according to claim 2, wherein
the cone is collapsible.

20. The dental X-ray device according to claim 2, further comprising:
a display section provided for the body.

21. The dental X-ray device according to claim 20, wherein
the display section has a visual recognition range overlapping with an area with which the dental lighting section irradiates with light.

22. The dental X-ray device according to claim 2, further comprising:
a visible light image capture section configured to capture an image of a site on which the X rays emitted from the X-ray emitter section are incident.

23. The dental X-ray device according to claim 20, further comprising:
a visible light image capture section configured to capture an image of a site on which the X rays emitted from the X-ray emitter section are incident, wherein
the display section is configured to display the image.
